(19) United States
(12) Patent Application Publication
(10) Pub. No.: US 2025/0255356 A1
(43) Pub. Date: Aug. 14, 2025

(54) LOWER BODY GARMENTS FOR WOMEN AND METHODS OF PREPARING SAME

(71) Applicant: Jessica Krupa, West Palm Beach, FL (US)

(72) Inventor: Jessica Krupa, West Palm Beach, FL (US)

(21) Appl. No.: 19/190,788

(22) Filed: Apr. 28, 2025

Related U.S. Application Data
(63) Continuation of application No. 17/359,943, filed on Jun. 28, 2021, now abandoned.

(51) Int. Cl. A41B 9/04; A41B 9/00; D04B 1/24

(52) U.S. Cl. CPC A41B 9/04; A41B 9/004; D04B 1/243; D10B 2501/02

(57) ABSTRACT

A lower body garment for women includes a main body fabric and a gusset fabric. The main body fabric has an inner and an outer side, a front and a rear end and a first vertical axis having length L1, and curved side ends having width W1. The gusset fabric has an inner and an outer side, a front and a rear end and a second vertical axis having length L2, and curved side ends having width W2. Moreover, the gusset fabric is secured to the main body fabric such that: the outer side of gusset fabric is arranged to face the inner side of main body fabric. The inner side of the gusset fabric is operable to cover a vulva, and wherein when in use, length L2 corresponds to a length of the vulva.

### CROSS REFERENCE TO RELATED APPLICATIONS

The present application is a continuation of, and claims priority from, U.S. patent application Ser. No. 17/359,943, filed on 28 Jun. 2021, which is incorporated herein by reference in its entirety.

### TECHNICAL FIELD

The aspects of the disclosed embodiments relate generally to undergarments and more specifically, lower body garments, comprising a gusset, for women.

### BACKGROUND

Every year, millions of women suffer from various gynaecological problems including, but not limited to, urinary tract infections (UTIs), vaginal infections including yeast and bacterial vaginosis (BV), vulvitis, itching, irritation and overall discomfort of the genitalia. Despite several medical advancements and educational programmes in this regard, the situation of female health and hygiene is failing to address this in the developed and the developing nations alike. While wearing panties to cover the external genitalia seems to be a solution, the problems arising from the use of panties itself have not been widely addressed.

Conventionally, available panties expose the external genitalia, especially vulva, to chemically treated synthetic fabrics found in panties. Notably, such synthetic fabrics may cause one or more of the aforementioned gynaecological problems. Moreover, the conventional panties lack enough breathability to prevent the growth of bacteria and yeast that cause most of the genital infections.

The undergarment industry introduced panty gussets as a solution to the breathability lacking in the conventional panties, by sewing fabric liners to the crotch portion of the panties to protect against synthetic materials used in the conventional panties. However, the available gussets don't cover the entire vulva, and expose the vulva to chemically treated synthetic materials such as polyester, nylon, spandex and other synthetic materials, thereby making the genitalia more prone to trapping heat, moisture and bacteria that can lead to infections. Moreover, use of products such as absorbent pads, disposable panty liners, and the like, over the conventional gusset could be a temporary solution. However, such disposable articles require frequent changing, as they can still lead to gynaecological issues. Moreover, while in use, such disposable articles expose the external genitalia to harmful chemicals and create an environment inside the undergarment which propagates yeast and bacterial infections. Furthermore, such disposable products are eventually dumped in a landfill thus creating another big challenge of waste management and environmental pollution.

Therefore, in light of the foregoing discussion, there exists a need to overcome the aforementioned drawbacks associated with existing approaches for improving feminine health and hygiene by using gussets that suitably cover the entire external genitalia, especially the vulva of the woman to prevent these issues.

### SUMMARY

The aspects of the disclosed embodiments are directed to a lower body garment for a woman and method of preparing the same. In one embodiment, the aspects of the disclosed embodiments seek to provide a solution to the existing problems of lower body garments that fail to provide a breathable space for the crotch portion of the female torso. An aim of the aspects of the disclosed embodiments is to provide a solution that overcomes at least partially the problems encountered in prior art, and provides a lower body garment comprising a gusset made from an organic, breathable, absorbent fabric that is configured to cover the vulva of the woman.

In one aspect, the aspects of the disclosed embodiments provide a lower body garment for a woman, the garment comprising
a main body fabric having an inner side, an outer side opposite the inner side, a front end, a rear end opposite the front end, a symmetrical vertical axis having a length L1 between the front end and the rear end, and a pair of curved side ends each on either side of the symmetrical vertical axis having a width W1 therebetween; and
a gusset fabric having an inner side, an outer side opposite the inner side, a front end, a rear end opposite the front end, a symmetrical vertical axis having a length L2 between the front end and the rear end, and a pair of curved side ends each on either side of the symmetrical vertical axis having a width W2 therebetween, the gusset fabric secured to the main body fabric such that:
   the outer side of the gusset fabric is arranged to face the inner side of the main body fabric, wherein the front end, the rear end, and the pair of curved side ends of the gusset fabric corresponds to the main body fabric, and
   the front end, the rear end, and the pair of curved side ends of the gusset fabric are secured to the main body fabric at seams of the gusset fabric,
      wherein the inner side of the gusset fabric is operable to cover the vulva of the woman, when in use, and wherein the length L2 corresponds to a length of the vulva.

In another aspect, the aspects of the disclosed embodiments provide a method of preparing a lower body garment for a woman, the method comprising
obtaining a main body fabric having an inner side, an outer side opposite the inner side, a front end, a rear end opposite the front end, a symmetrical vertical axis having a length L1 between the front end and the rear end, and a pair of curved side ends each on either side of the symmetrical vertical axis having a width W1 therebetween;
obtaining a gusset fabric having an inner side, an outer side opposite the inner side, a front end, a rear end opposite the front end, and a symmetrical vertical axis having a length L2 between the front end and the rear end, and a pair of curved side ends each on either side of the symmetrical vertical axis having a width W2 therebetween; and
securing the gusset fabric to the main body fabric such that:
   the outer side of the gusset fabric is arranged to face the inner side of the main body fabric, wherein the front end, the rear end, and the pair of curved side ends of the gusset fabric correspond to the main body fabric, and
   the front end, the rear end, and the pair of curved side ends of the gusset fabric are secured to the main body fabric at seams of the gusset fabric,
      wherein the inner side of the gusset fabric is operable to cover the vulva of the woman, when in use, and wherein the length L2 corresponds to a length of the vulva.

The aspects of the disclosed embodiments substantially eliminate or at least partially address the aforementioned problems in the prior art, and enable a robust lower body garment that provides a better coverage of the external genitalia, allows space for the crotch portion of the female torso and enhances female health and hygiene. Additionally, beneficially, the gusset fabric is secured to a main body fabric of the lower body garment to provide a durable and robust product. Moreover, the effective securing of the gusset fabric to the main body fabric throughout the edges of the gusset fabric ensures that the gusset fabric does not move, shift or roll during use or washing, for example, thereby providing for a reusable product.

Additional aspects, advantages, features and objects of the aspects of the disclosed embodiments would be made apparent from the drawings and the detailed description of the illustrative embodiments construed in conjunction with the appended claims that follow.

It will be appreciated that features of the aspects of the disclosed embodiments are susceptible to being combined in various combinations without departing from the scope of the aspects of the disclosed embodiments as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the aspects of the disclosed embodiments, exemplary constructions of the disclosure are shown in the drawings. However, the aspects of the disclosed embodiments are not limited to specific methods and instrumentalities disclosed herein. Moreover, those skilled in the art will understand that the drawings are not to scale. Wherever possible, like elements have been indicated by identical numbers.

The aspects of the disclosed embodiments will now be described, by way of example only, with reference to the following diagrams wherein:

FIGS. 1A, 1B, 1C, and 1D are perspective views of a lower body garment, implemented as a low-rise hipster, for a woman, in accordance with the aspects of the disclosed embodiments;

FIGS. 2A and 2B are perspective views of a lower body garment, implemented as a high-rise hipster, for a woman, in accordance with the aspects of the disclosed embodiments;

FIGS. 3A and 3B are perspective views of a lower body garment, implemented as a mid-rise bikini, for a woman, in accordance with the aspects of the disclosed embodiments;

FIGS. 4A and 4B are perspective views of a lower body garment, implemented as a high-rise bikini, for a woman, in accordance with the aspects of the disclosed embodiments;

FIGS. 5A and 5B are perspective views of a lower body garment, implemented as a low-rise thong, for a woman, in accordance with the aspects of the disclosed embodiments;

FIGS. 6A. and 6B are perspective views of a lower body garment, implemented as a high-rise thong, for a woman, in accordance with the aspects of the disclosed embodiments; and

FIG. 7 is a flowchart of steps of a method of preparing a lower body garment for a woman, in accordance with an aspect of the disclosed embodiments.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates exemplary aspects of the disclosed embodiments and ways in which they can be implemented. Although some modes of carrying out the aspects of the disclosed embodiments have been disclosed, those skilled in the art would recognize that other embodiments for carrying out or practising the aspects of the disclosed embodiments are also possible.

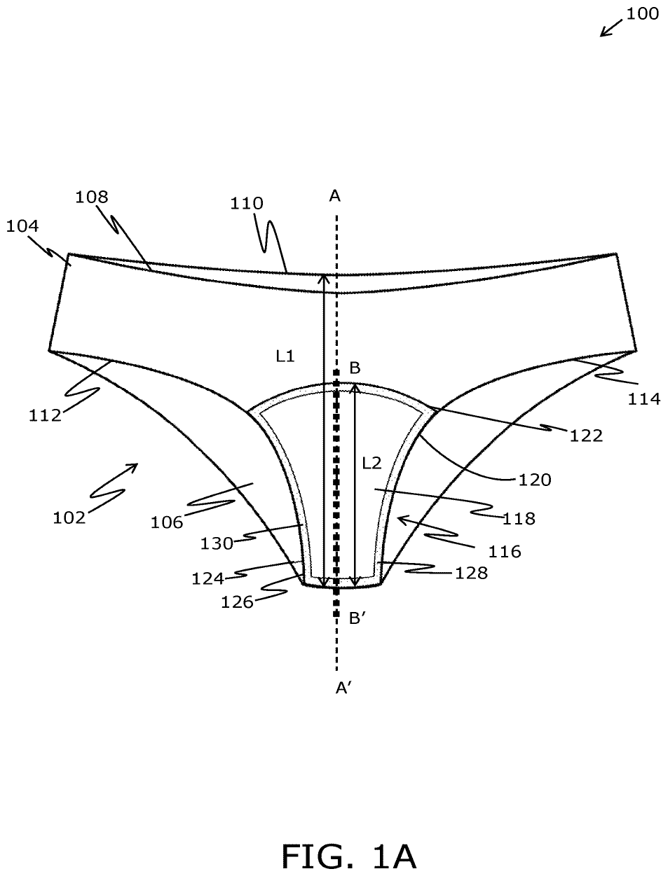

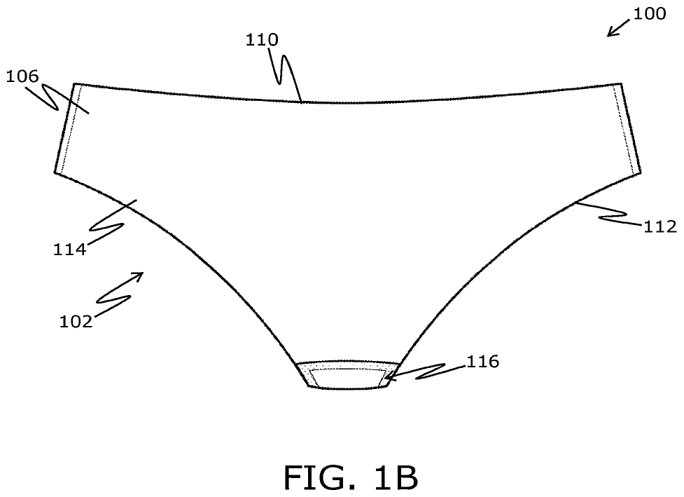

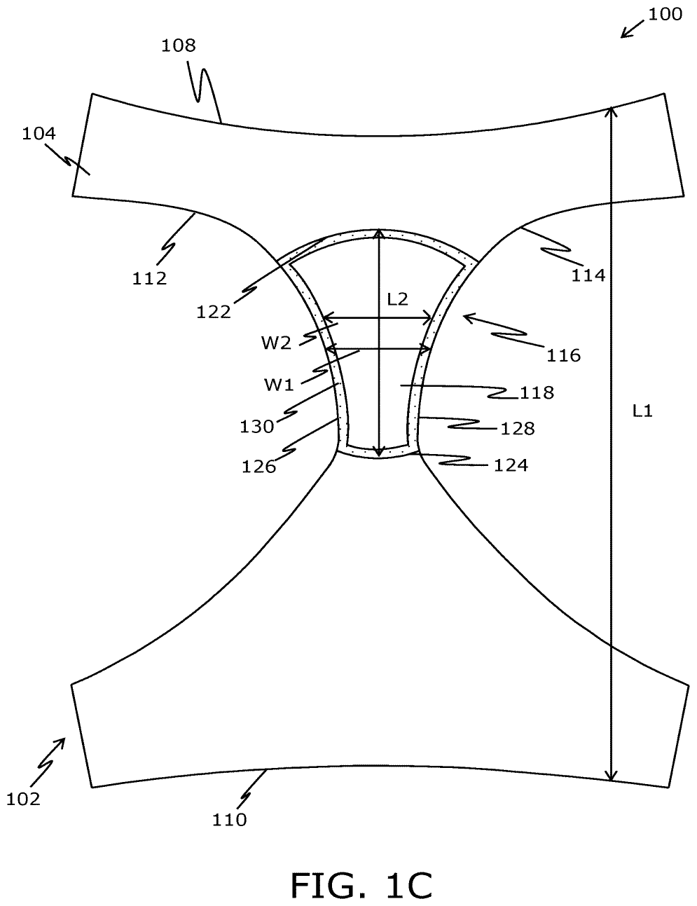

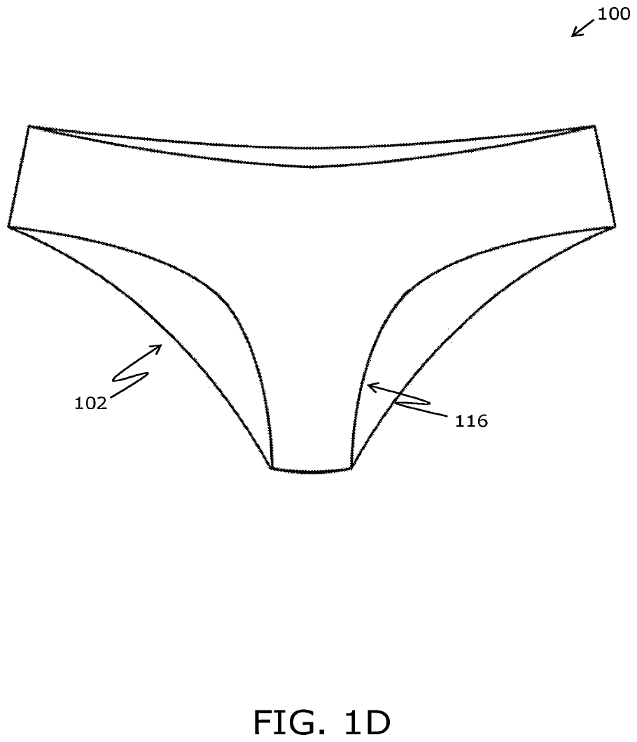

Referring to FIGS. 1A, 1B, 1C, and 1D, illustrated are perspective views of a lower body garment 100, implemented as a low-rise hipster, for a woman, in accordance with an aspect of the disclosed embodiments. As shown, FIG. 1A depicts a front view of the lower body garment 100 of an inside surface thereof, FIG. 1B depicts a rear view of the lower body garment 100 of an inside surface thereof, FIG. 1C depicts an inner surface of the lower body garment 100, and FIG. 1D depicts a front view of the lower body garment 100 from an outside surface thereof. Beneficially, the lower body garment 100 is a robust, durable and reusable product that enables maintaining female health and hygiene by completely covering the vulva of the female torso.

Throughout the present disclosure, the term “lower body garment” 100 as used herein refers to a close-fitting (namely, form-fitting) garment worn usually next to skin around the pelvic region (between abdomen and thighs) of a human torso, especially that of a woman. The lower body garment 100 typically comprises at least a waistband that runs along the waist of the woman, a crotch panel to cover at least partially external genitals of the woman, and a pair of leg openings. Optionally, the external genitals include a vulva. The vulva typically includes a clitoris, a labia majora, a labia minora, a vestibule, a urethral meatus, an introitus and a perineum. Optionally, the lower body garment 100 could have different forms and sizes to suit different body-types and purposes. Optionally, the lower body garment 100 includes various panty styles, such as a boyshort, a hipster, a thong, a bikini, a G-string, a tanga, and the like, in various rises including a low-rise, a mid-rise, and a high-rise. Moreover, optionally, the lower body garment 100 could be used in other lower body garments, such as a shapewear, activewear, and everyday wear such as leggings, pantyhose, stockings, knickers, underpants, and the like. It will be appreciated that the lower body garment 100 may be available in different sizes ranging from women's extra-extra-small (XXS), extra-small (XS) to extra-large (XL), extra-extra-large (XXL), and women's plus sizes from 1X to 5X. In an example, the size of the lower body garment 100 may be any of: extra-extra-small (XXS), extra-small (XS), Small (S), Medium (M), large (L), extra-large (XL), extra-extra-large (XXL), 1X, 2X, 3X, 4X and 5X, to suit women of different sizes.

The lower body garment 100 comprises a main body fabric 102 having an inner side 104, an outer side 106 opposite the inner side 104, a front end 108, a rear end 110 opposite the front end 108, a symmetrical vertical axis A-A′ having a length L1 between the front end 108 and the rear end 110, and a pair of curved side ends 112, 114 each on either side of the symmetrical vertical axis A-A′ having a width W1 therebetween. Throughout the present disclosure, the term “main body fabric” 102 as used herein refers to a primary piece of fabric (namely a woven, non-woven or knitted cloth) that forms the base material of the lower body garment 100. Typically, the main body fabric 102 forms the lower body garment 100. In this regard, the main body fabric 102 is cut and stitched and/or bonded to result in the waistband (namely, a portion of the main body fabric 102 that encircles the waist of the wearer, i.e. the woman), the crotch panel (namely, a portion of the main body fabric 102 that covers external genitalia of the wearer, i.e. the woman), and the pair of leg openings (namely, parallel openings in the main body fabric 102 that allow arranging the crotch panel of the main body fabric 102 in between the legs of the wearer, i.e. the woman) of the lower body garment 100. It will be appreciated that, when in use, the inner side 104 lies next to the skin of the torso and is expected to be a skin-friendly fabric, while the outer side 106 contributes mainly to the aesthetics of the lower body garment 100. Moreover, the front end 108 and rear end 110 correspond respectively to a front anatomical structure and a rear anatomical structure of the user, that is the woman using the said lower body garment 100. Moreover, the lower body garment 100 comprises the crotch panel at a pre-defined distance from the waistband of the lower body garment 100 extending from at least a part of the front end 108 up to a part of the rear end 108. It will be appreciated that the main body garment 102 is vertically symmetrical along the entire length of the main body garment 102.

Optionally, the main body fabric 102 is selected from a group comprising: organic cotton, cotton, cotton spandex, bamboo, bamboo spandex, hemp, linen, muslin, synthetic, wool, wool blend, elastane, rayon, nylon, satin, silk, lace, organza, jersey, net, charmeuse, spandex, polyester, micro modal, merino wool, or any combination thereof. It will be appreciated that the main body fabric 102 is selected to provide the lower body garment 100 with the desired characteristics, such as for example both aesthetic and comfort.

Moreover, the garment 100 comprises a gusset fabric 116 having an inner side 118, an outer side 120 opposite the inner side 118, a front end 122, a rear end 124 opposite the front end 122, a symmetrical vertical axis B-B′ having a length L2 between the front end 122 and the rear end 124, and a pair of curved side ends 126, 128 each on either side of the symmetrical vertical axis B-B′ having a width W2 therebetween. Throughout the present disclosure, the term “gusset fabric” 116 as used herein refers to a secondary (relatively smaller as compared to the main body fabric 102) piece of fabric (namely a woven, non-woven or knitted cloth) that is arranged to line the crotch panel of the lower body garment 100. Optionally, the gusset fabric 116 is a single layer fabric made of a single type (or length) of fabric. The gusset fabric 116 is expected to be a skin-friendly, breathable and absorbent fabric that is optimum for feminine health and hygiene. Beneficially, using a fabric woven from organic cotton fibres that are breathable and absorbent that prevent growth of bacteria and yeast in the dark moist environments of the female intimate areas that are the leading causes of majority of urinary tract infections, bacterial infections, vulvitis, smells, odours, itching, irritation, and an overall misery of the genitalia. Additionally, beneficially, gusset fabric 116 made from organic, breathable, absorbent fibres promote female health and hygiene.

Optionally, the gusset fabric 116 is selected from a group comprising: organic cotton, cotton, linen, muslin, bamboo, hemp, wool, wool blend, elastane, or any combination thereof. It will be appreciated that the aforesaid fabrics for making the gusset fabric 116 are made of organic and breathable fibres with a high absorption quality. Optionally, the gusset fabric 116 is made from a 100% organic cotton fibre. Moreover, the gusset fabric 116 may be made using any fabric combination that is optimum for feminine health and hygiene. Moreover, the gusset fabric 116 may be selected from fabrics that are not chemically treated (namely, chemical-free fabrics), and therefore suitable for preventing gynaecological problems.

Furthermore, the gusset fabric 116 is secured to the main body fabric 102 such that the outer side 120 of the gusset fabric 116 is arranged to face the inner side 104 of the main body fabric 102, wherein the front end 122, the rear end 124, and the pair of curved side ends 126, 128 of the gusset fabric 116 corresponds to the main body fabric 102. It will be appreciated that the term “correspond” as used herein refers to an arrangement of the gusset fabric 116 and the main body fabric 102 such that the inner side 118 and the outer side 120 of the gusset fabric 116 are parallel to the inner side 104 and the outer side 106 of the main body fabric 102, the front end 122 of the gusset fabric 116 is parallel to the front end 108 of the main body fabric 102, the rear end 124 of the gusset fabric 116 is parallel to the rear end 110 of the main body fabric 102, the symmetrical vertical axis B-B′ of the gusset fabric 116 is parallel to the symmetrical vertical axis A-A′ of the main body fabric 102, and the pair of curved side ends 126, 128 of the gusset fabric 116 is parallel to the pair of curved side ends 112, 114 of the main body fabric 102. It will be appreciated that the term “correspond” does not imply that the aforesaid sides or ends coincide, i.e. the front end 122 of the gusset fabric 116 with the front end 108 of the main body fabric 102, the rear end 124 of the gusset fabric 116 with the rear end 110 of the main body fabric 102, and the curved side ends 126, 128 of the gusset fabric 116 with the curved side ends 112, 114 of the main body fabric 102.

Moreover, the pair of curved side ends 126, 128 of the gusset fabric 116 are secured to the main body fabric 102 at seams 130 of the gusset fabric 102. The term “seam” 130 as used herein refers to edges of the gusset fabric 116 corresponding to the front end 122, the rear end 124, and the pair of curved side ends 126, 128. Notably, the seams 130 of the gusset fabric 116 are equal throughout the gusset fabric 116. Alternatively, the width of seams 130 may vary on the front end 122, the rear end 124, and/or the pair of curved side ends 126, 128, based on the grading of the lower body garment size. Securing is typically performed at the seams 130.

Throughout the present disclosure, the term “secured” as used herein refers to attaching firmly in a manner to prevent the attached parts from moving against each other. Optionally, the gusset fabric 116 is secured to the main body fabric 102 along at least one of: the front end 122 of the gusset fabric 116, the rear end 124 of the gusset fabric 116, the pair of curved side ends 126, 128 of the gusset fabric 116, or a combination thereof. Preferably, the gusset fabric 116 is secured to the main body fabric 116 along all of ends of the gusset fabric 116, i.e. the front end 122, the rear end 124, the pair of curved side ends 126, 128, to enable a robust securing of the gusset fabric 116 to the main body fabric 116. Beneficially, securing the gusset fabric 116 at all its four ends to the main body fabric 102 prevents the gusset fabric 116 from rolling or displacing during use, or moving or shifting during wearing or while washing. Additionally, beneficially, the sideways securing of the gusset fabric 116 and the main body fabric 102 enhances the comfort level associated with the gusset fabric 116. However, alternatively, optionally, securing could be performed throughout the gusset fabric 116 and the main body fabric 102, i.e. besides at all its four ends thereof, the gusset fabric 116 is secured to the main body fabric 102 at its middle portion also, i.e. portion covered between the pair of curved side ends 126, 128 and the front end 122 and the rear end 124. It will be appreciated that securing of the gusset fabric 116 to the main body fabric 102 as mentioned above ensures a durable final product. Moreover, securing of the gusset fabric 116 to the main body fabric 102 as mentioned above ensures only the skin-friendly fabric, i.e. the gusset fabric 116, to be next to the vulva of the woman. Furthermore, securing the gusset fabric 116 to the main body fabric 102 as mentioned above results in a more secure lower body garment 100, providing a more comfortable feel and a better fit experience by the woman.

Optionally, the gusset fabric 116 is secured to the main body fabric 102 using at least one of: a stitch, a bond, an adhesive. Optionally, the means for securing is selected based on the types of gusset fabric 116 and the main body fabric 102. The term “stitch” typically refers to joining the gusset fabric 116 and the main body fabric 102 using a thread and needle (by hands or a sewing machine), for example. The term “bond” typically relates to a stitch-less joining (namely, fusing, gluing or adhering) of the gusset fabric 116 and the main body fabric 102 using heat, ultrasonic waves, for example. Typically, securing using bond is suitable for thermoplastic fabrics, however, bonding, using an additional adhesive in between the two layers of fabric, such as the gusset fabric 116 and the main body fabric 102, could be used universally for all types of fabrics. The term “adhesive” refers to a fabric-specific glue and/or tape that is used between two layers of fabric, i.e. the gusset fabric 116 and the main body fabric 102, and set by application of pressure thereon. Beneficially, the securing of the gusset fabric 116 to the main body fabric 102 does not result in a visible impression on the outer surface of the lower body garment 100.

Moreover, the inner side 118 of the gusset fabric 116 is operable to cover vulva (not shown) of the woman, when in use, and wherein the length L2 corresponds to a length of the vulva. Optionally, when in use, the gusset fabric 116 is operable to cover vulva with the front end 122 ending at the mons pubis. The length L2 of the gusset fabric 116 increases corresponding to the size of the lower body garment 100 in order to provide a fuller coverage to the vulva. It will be appreciated that the length L2 of the gusset fabric 116 could be of different measurements in order to fit on the lower body garment 116 ranging typically from XXS, XS, S, M, L, XL, XXL, 1X 2X, 3X, 4X, 5X, and all other women's sizes. Moreover, the width W2 of the gusset fabric 116 could be of different measurements in order to fit on the lower body garment 116 ranging typically from XXS, XS, S, M, L, XL, XXL, 1X 2X, 3X, 4X, 5X, and all other women's sizes.

Optionally, the length L2 is smaller than the length L1 and is at least 5 inches and curved at the front end 122 of the gusset fabric 116 to cover the vulva. Typically, the length L2 is in a range from 5 to 12 inches. The length L2 may for example be from 5, 6, 7, 8, 9, 10 or 11 inches up to 6, 7, 8, 9, 10, 11 or 12 inches. It will be appreciated that the above-mentioned range may include a range of sizes such as 5¼, 6½, 7¾, 8⅕, 10½, and the like. Beneficially, the curved shape of the front end 122 of the gusset fabric 116 enables complete protection of the vulva.

Optionally, the gusset fabric is bonded on the inner side thereof that covers the vulva. The inner side 118 of the gusset fabric 116 ensures proper comfort to the user, when the lower body garment 100 is worn by the user.

Optionally, the widths W1 and W2 are equal. It will be appreciated that the pair of curved ends 126, 128 of the gusset fabric 116 and the pair of curved ends 112, 114 of the main body fabric 102 coincide while bonding thereof. Optionally, the width W2 could be marginally smaller, such as by ±0.05-0.15 inch than the width W1, however, the curved ends 126, 128 of the gusset fabric 116 and the pair of curved ends 112, 114 are secured together. It will be appreciated that the marginally smaller width W2 provides a tolerance limit from the edge to edge of the seams of the gusset fabric 116 and the main body fabric 102. In an example, a tolerance could be of 0.15 inch (or ⅛ inch).

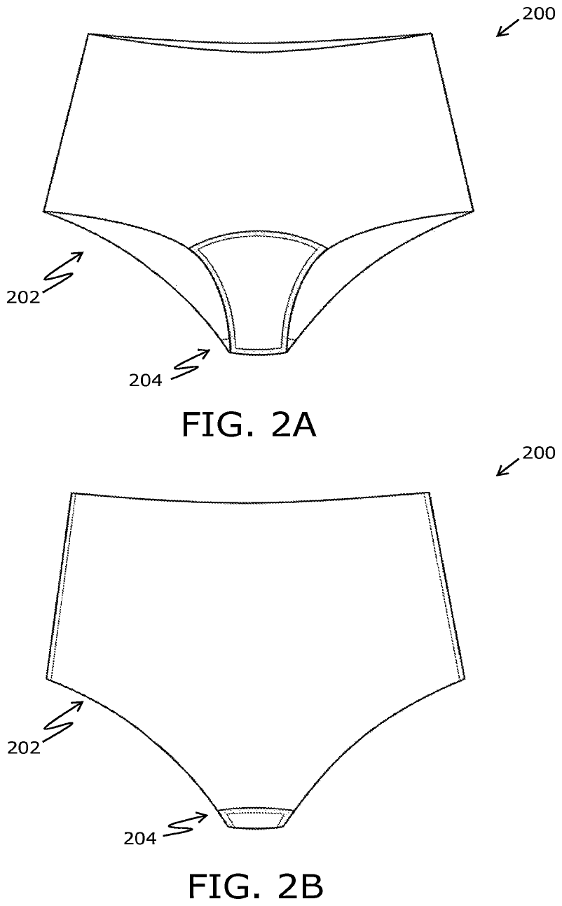

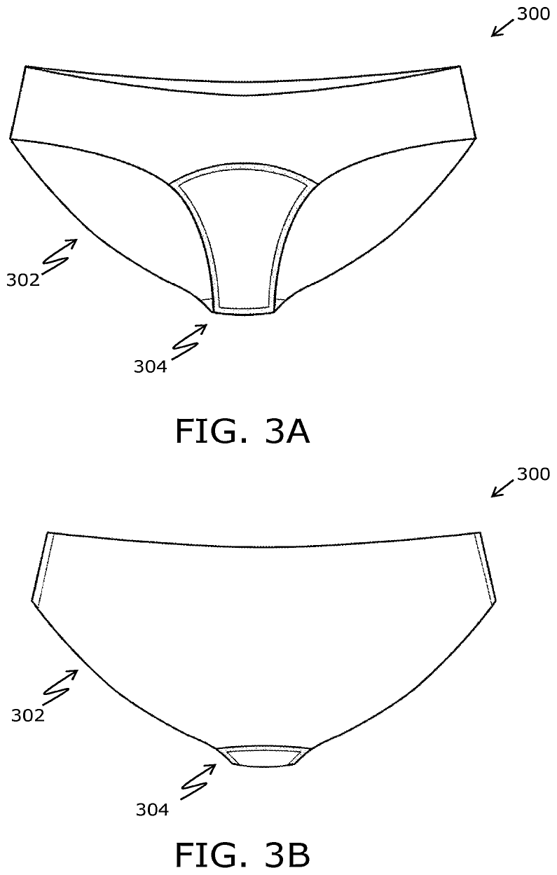

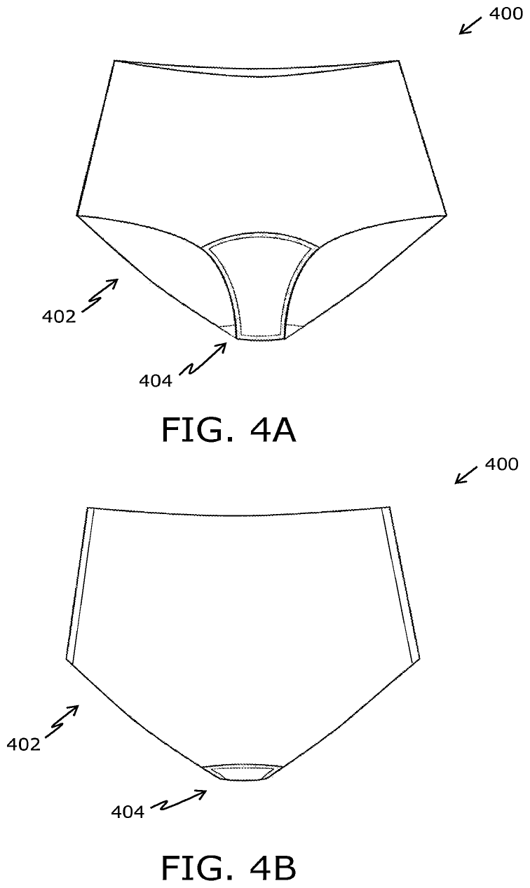

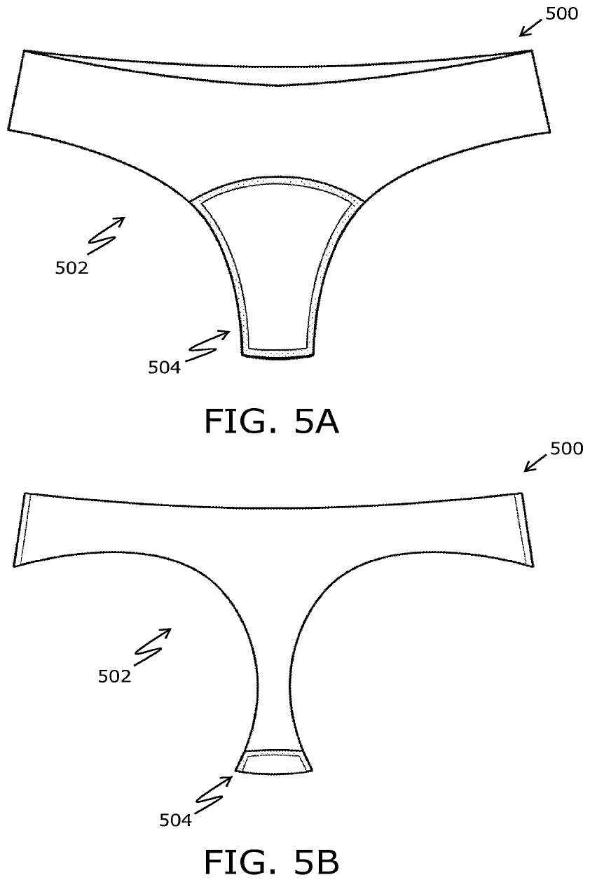

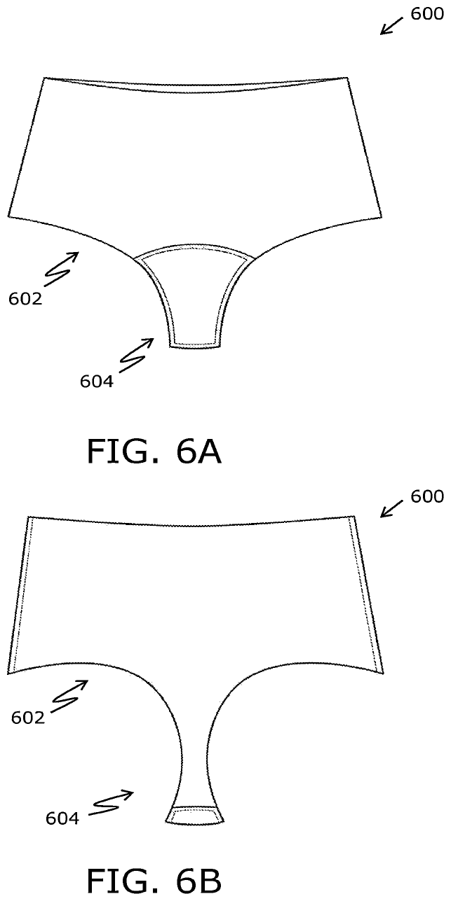

Referring to FIGS. 2A, 2B, 3A, 3B, 4A, 4B, 5A, 5B, 6A and 6B, illustrated are perspective views of different styles of a lower body garment, i.e. 200, 300, 400, 500 and 600, for a woman, in accordance with various aspect of the disclosed embodiments. As shown, FIGS. 2A, 3A, 4A, 5A and 6A depict a front view of an inside surface of the lower body garment, i.e. 200, 300, 400, 500 and 600, respectively, and the FIGS. 2B, 3B, 4B, 5B and 6B depict the back view of an inside surface of the lower body garment, i.e. 200, 300, 400, 500 and 600. Moreover, the lower body garment is implemented as a high-rise hipster 200, a mid-rise bikini 300, a high-rise bikini 400, a low-rise thong 500 and a high-rise thong 600. It will be appreciated that the lower body garment, i.e. 200, 300, 400, 500 and 600 are similar in embodiment to the lower body garment 100 of FIG. 1. Each of the lower body garment, i.e. 200, 300, 400, 500 and 600, comprises a main body fabric 202, 302, 402, 502, 602 and the gusset fabric 204, 304, 404, 504, 604, the gusset fabric 204, 304, 404, 504, 604 secured to the main body fabric 202, 302, 402, 502, 602 at seams of the gusset fabric 204, 304, 404, 504, 604.

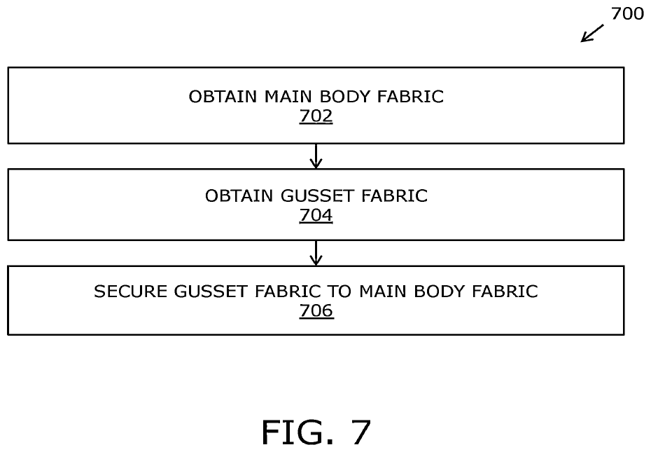

Referring to FIG. 7, illustrated is a flowchart 700 of steps of a method of preparing a lower body garment (such as the lower body garment 100, 200, 300, 400, 500, 600) for a woman, in accordance with an aspect of the disclosed embodiments. At step 702, a main body fabric 102, 202, 302, 402, 502, 602, having an inner side 104, an outer side 106 opposite the inner side 104, a front end 108, a rear end 110 opposite the front end 108, a symmetrical vertical axis A-A′ having a length L1 between the front end 108 and the rear end 110, and a pair of curved side ends 112, 114 each on either side of the symmetrical vertical axis A-A′ having a width W1 therebetween, is obtained.

At step 704, a gusset fabric 116, 204, 304, 404, 504, 604, having an inner side 118, an outer side 120 opposite the inner side 118, a front end 122, a rear end 124 opposite the front end 122, and a symmetrical vertical axis B-B′ having a length L2 between the front end 122 and the rear end 122, and a pair of curved side ends 126, 128 each on either side of the symmetrical vertical axis B-B′ having a width W2 therebetween, is obtained.

At step 706, the gusset fabric 116, 204, 304, 404, 504, 604 is secured to the main body fabric 102, 202, 302, 402, 502, 602 such that the outer side 120 of the gusset fabric 116, 204, 304, 404, 504, 604 is arranged to face the inner side 104 of the main body fabric 102, 202, 302, 402, 502, 602, wherein the front end 122, the rear end 124, and the pair of curved side ends 126, 128 of the gusset fabric 116, 204, 304, 404, 504, 604 correspond to the main body fabric 102, 202, 302, 402, 502, 602, and the front end 122, the rear end 124, and the pair of curved side ends 126, 128 of the gusset fabric 116, 204, 304, 404, 504, 604 are secured to the main body fabric 102, 202, 302, 402, 502, 602 at seams 130 of the gusset fabric 116, 204, 304, 404, 504, 604, wherein the inner side 118 of the gusset fabric 116, 204, 304, 404, 504, 604 is operable to cover the vulva of the woman, when in use, and wherein the length L2 corresponds to a length of the vulva.

The steps 702, 704 and 706 are only illustrative and other alternatives can also be provided where one or more steps are added, one or more steps are removed, or one or more steps are provided in a different sequence without departing from the scope of the claims herein.

Various embodiments and variants disclosed above, with respect to the aforementioned system (100, 200, 300, 400, 500, 600), apply mutatis mutandis to the method. Beneficially, the method of the present disclosure ensures obtaining chemical-free fabrics for the gusset fabric 116, 204, 304, 404, 504, 604, thereby preventing myriad gynaecological problems. Moreover, the design and securing of the gusset fabric 116, 204, 304, 404, 504, 604 to the main body fabric 102, 202, 302, 402, 502, 602 provides full coverage of the female genitalia to enhance female health and hygiene, while still providing a robust product that does not shift or move while in use or washing. Additionally, beneficially, the methods of securing the gusset fabric 116, 204, 304, 404, 504, 604 to the main body fabric 102, 202, 302, 402, 502, 602 ensures a more secure garment resulting in better comfort and fit experience to the user of the different styles of lower body garment 100, 200, 300, 400, 500, 600.

Optionally, the gusset fabric 116, 204, 304, 404, 504, 604 and the main body fabric 102, 202, 302, 402, 502, 602 are secured using at least one of: a stitch, a bond, an adhesive.

Optionally, the gusset fabric 116, 204, 304, 404, 504, 604 is secured to the main body fabric 102, 202, 302, 402, 502, 602 along at least one of: the front end 122 of the gusset fabric 116, 204, 304, 404, 504, 604, the rear end 124 of the gusset fabric 116, 204, 304, 404, 504, 604, the pair of curved side ends 126, 128 of the gusset fabric 116, 204, 304, 404, 504, 604, or a combination thereof.

Optionally, the gusset fabric 116, 204, 304, 404, 504, 604 is bonded on the inner side 118 thereof that covers the vulva.

Optionally, the main body fabric 102, 202, 302, 402, 502, 602 is selected from a group comprising: organic cotton, cotton, cotton spandex, bamboo, bamboo spandex, hemp, linen, muslin, synthetic, wool, wool blend, elastane, rayon, nylon, satin, silk, lace, organza, jersey, net, charmeuse, spandex, polyester, micro modal, merino wool, or any combination thereof.

Optionally, the gusset fabric 116, 204, 304, 404, 504, 604 is selected from organic cotton, cotton, linen, muslin, bamboo, hemp, wool, wool blend, elastane, or any other fabric combination.

Optionally, the length L2 is smaller than the length L1 and is at least 5 inches and curved at the front end 122 of the gusset fabric 116, 204, 304, 404, 504, 604 to cover the vulva.

Optionally, the widths W1 and W2 are equal.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A lower body garment for a woman, the garment comprising:
a main body fabric having an inner side, an outer side opposite the inner side, a front end, a rear end opposite the front end, a symmetrical vertical axis having a length L1 between the front end and the rear end, and a pair of curved side ends each on either side of the symmetrical vertical axis having a width W1 therebetween; and
a gusset fabric having an inner side, an outer side opposite the inner side, a front end, a rear end opposite the front end, a symmetrical vertical axis having a length L2 between the front end and the rear end, and a pair of curved side ends each on either side of the symmetrical vertical axis having a width W2 therebetween, wherein the length L2 is smaller than the length L1 and is at least 5 inches and wherein the front end of the gusset fabric is curved and configured to cover a vulva of the woman, and wherein the width W2 is ±0.05-0.15 inch than the width W1, the gusset fabric secured to the main body fabric such that:
the outer side of the gusset fabric is arranged to face the inner side of the main body fabric, wherein the front end, the rear end, and the pair of curved side ends of the gusset fabric corresponds to the main body fabric, and
the front end, the rear end, and the pair of curved side ends of the gusset fabric are secured to the main body fabric at seams of the gusset fabric, wherein the gusset fabric is secured to the main body fabric along the front end of the gusset fabric. the rear end of the gusset fabric. the pair of curved side ends of the gusset fabric and at a middle portion of the gusset fabric. wherein the middle portion corresponds to a portion between the pair of curved side ends of the gusset fabric and the front end of the gusset fabric and the rear end of the gusset fabric;
wherein the inner side of the gusset fabric is operable to cover the vulva of the woman, and wherein when in use, the length L2 corresponds to a length of the vulva.

2. The lower body garment according to claim 1, wherein the gusset fabric is secured to the main body fabric using at least one of: a stitch, a bond, an adhesive.

3. The lower body garment according to claim 1, wherein the gusset fabric is bonded on the inner side thereof that covers the vulva.

4. The lower body garment according to claim 1, wherein the main body fabric is selected from a group comprising: organic cotton, cotton, cotton spandex, bamboo, bamboo spandex, hemp, linen, muslin, synthetic, wool, wool blend, elastane, rayon, nylon, satin, silk, lace, organza, jersey, net, charmeuse, spandex, polyester, micro modal, merino wool, or any combination thereof.

5. The lower body garment according to claim 1, wherein the gusset fabric is selected from a group comprising: organic cotton, cotton, linen, muslin, bamboo, hemp, wool, wool blend, elastane, or any combination thereof.

6. A method for preparing a lower body garment for a woman, the method comprising:
obtaining a main body fabric having an inner side, an outer side opposite the inner side, a front end, a rear end opposite the front end, a symmetrical vertical axis having a length L1 between the front end and the rear end, and a pair of curved side ends each on either side of the symmetrical vertical axis having a width W1 therebetween;
obtaining a gusset fabric having an inner side, an outer side opposite the inner side, a front end, a rear end opposite the front end, and a symmetrical vertical axis having a length L2 between the front end and the rear end, and a pair of curved side ends each on either side of the symmetrical vertical axis having a width W2 therebetween. wherein the length L2 is smaller than the length L1 and is at least 5 inches and wherein the front end of the gusset fabric is curved and configured to cover a vulva of the woman. and wherein the width W2 is ±0.05-0.15 inch than the width W1; and
securing the gusset fabric to the main body fabric such that:
the outer side of the gusset fabric is arranged to face the inner side of the main body fabric, wherein the front end, the rear end, and the pair of curved side ends of the gusset fabric correspond to the main body fabric, and
the front end, the rear end, and the pair of curved side ends of the gusset fabric are secured to the main body fabric at seams of the gusset fabric, wherein the gusset fabric is secured to the main body fabric along the front end of the gusset fabric. the rear end of the gusset fabric. the pair of curved side ends of the gusset fabric and at a middle portion of the gusset fabric. wherein the middle portion corresponds to a portion between the pair of curved side ends of the gusset fabric and the front end of the gusset fabric and the rear end of the gusset fabric;
wherein the inner side of the gusset fabric is operable to cover the vulva of the woman, and wherein when in use, the length L2 corresponds to a length of the vulva.

7. The method according to claim 6, wherein the gusset fabric and the main body fabric are secured using at least one of: a stitch, a bond, an adhesive.

8. The method according to claim 6 wherein the gusset fabric is bonded on the inner side thereof that covers the vulva.

9. The method according to claim 6, wherein the main body fabric is selected from a group comprising: organic cotton, cotton, cotton spandex, bamboo, bamboo spandex, hemp, linen, muslin, synthetic, wool, wool blend, elastane, rayon, nylon, satin, silk, lace, organza, jersey, net, charmeuse, spandex, polyester, micro modal, merino wool, or any combination thereof.

10. The method according to claim 6, wherein the gusset fabric is selected from a group comprising: organic cotton, cotton, linen, muslin, bamboo, hemp, wool, wool blend, elastane or any other fabric combination.
